(19) United States
(12) Patent Application Publication
(10) Pub. No.: US 2009/0043390 A1
(43) Pub. Date: Feb. 12, 2009

(54) Parts Assembly and Part for a Prosthesis

(76) Inventor: Hans Jorg Meisel, Berlin (DE)

Correspondence Address: SUTHERLAND ASBILL & BRENNAN LLP, 999 PEACHTREE STREET, N.E., ATLANTA, GA, 30309, US

(21) Appl. No.: 10/596,158

(22) PCT Filed: Dec. 15, 2004
(86) PCT No.: PCT/DE04/02748

§ 371 Date: Sep. 29, 2006

(30) Foreign Application Priority Data
Dec. 22, 2003 (DE) 103 61 166.5
Jun. 9, 2004 (DE) 10 2004 027 986.1

(51) Int. Cl. A61F 2/44

(52) U.S. Cl. 623/17.16; 623/17.11

(57) ABSTRACT

The invention relates to a parts assembly for a prosthesis, particularly a cervical spine intervertebral disc prosthesis, comprising two base parts (1, 2), which are coupled to one another in an articulated manner by means of coupling parts (11, 12) formed on said base parts. These base parts, together with an associated coupling part, are provided as a single piece. The base parts and the coupling parts are made of one material, which is selected from the following group of materials: polyetherketone (PEK) polyetheretherketone (PEEK), polyacryletherketone (PAEK), polyetherketoneketone (PEKK), polyetherketoneetherketoneketone (PEKEKK) and polyetherketoneetherketone (PEKEK).

The invention relates to a parts assembly and to a part for a prosthesis, particularly a cervical spine intervertebral disc prosthesis.

### BACKGROUND OF THE INVENTION

Prostheses based on one or more parts are used to support the functionality of, or even replace, parts of the bone skeleton, for example the spine or a joint.

Degenerative damage to the cervical spine which is associated with a herniated disc or compresses the spinal cord as a result of bone constriction is usually operated on ventrally when an operation is required. When operating in this way, the cervical disc usually has to be completely removed in order to relieve the pressure on the spinal cord and the nerve root. To date, this operative procedure has of course been associated with a loss of function of the affected vertebral motor segment. In order to prevent an additional loss of height of the disc, which can lead to an increase in degenerative and neurological changes, the ventral fusion operation has until now been the method of choice.

By using bone cement, iliac crest bone or cage-shaped spacers (so-called cages) adapted to the height of the disc, at least the height of the disc is reconstructed in this case, wherein a further aim of this procedure is to achieve bone fusion of the vertebral bodies of the affected vertebral motor segment. This has the disadvantage that the connecting vertebral motor segments are worse affected than before by the lifting effect of the fused vertebrae obtained after fusion, which promotes increasing degeneration in these connecting segments. When suitably indicated, a future aim of this treatment method is to retain the function of the segment by providing a full disc prosthesis.

Developments proposed to date for cervical spine intervertebral disc prostheses, which have led to market-ready products, are not very widely used in terms of clinical use since complicated operational procedures are required to implant them and these are associated with irreversible changes to the lower end plate and upper end plate of the affected vertebral bodies. For example, in order to use the prosthesis according to Bryan et al., parts of the adjoining vertebral bodies have to be removed in order on the one hand to fix the prosthesis in place but also to accommodate the relatively large height of the implant. The reason for the high size of this implant is the very complex structure, which performs a shock-absorbing function, and also the fact that the structure is composed of a large number of individual parts, which are made of different materials. Implantation takes a great deal of time and requires the use of more than 30 implantation instruments. The spontaneous fusion rate after implantation can be reduced by the post-operative administration of cortisone.

### SUMMARY OF THE INVENTION

It an object of the invention to provide an improved parts assembly and an improved part for a prosthesis, which are cost-effective and can be manufactured with as little complexity as possible.

According to the invention, this task is solved by a parts assembly according to independent claim 1 and by a part according to independent claim 11. Advantageous embodiments of the invention can be found in the dependent claims.

According to the invention, there is provided a parts assembly for a prosthesis, particularly a cervical spine intervertebral disc prosthesis, comprising two base parts, which are coupled to one another in an articulated manner by means of coupling parts formed on the base parts, wherein the base parts are in each case formed in one piece with an associated coupling part. By means of the design of the parts assembly for the prosthesis with two base parts, which are coupled to one another in an articulated manner, a mechanically simple structure is selected. Furthermore, it is provided that the base parts and the coupling parts are made of a material selected from the following group of materials: polyetherketone (PEK), polyetheretherketone (PEEK), polyacryletherketone (PAEK), polyetherketoneketone (PEKK), polyetherketoneetherketoneketone (PEKEKK) and polyetherketoneetherketone (PEKEK).

According to another aspect of the invention, there is provided a part for a prosthesis parts assembly, particularly a cervical spine intervertebral disc prosthesis part, comprising a base part and a coupling part formed on the base part for articulated coupling to another base part, wherein the base part and the coupling part are formed in one piece. Furthermore, it is provided that the base part and the coupling part are made of a material selected from the following group of materials: polyetherketone (PEK), polyetheretherketone (PEEK), polyacryletherketone (PAEK), polyetherketoneketone (PERK), polyetherketoneetherketoneketone (PEKEKK) and polyetherketoneetherketone (PEKEK).

Compared to known parts for cervical spine intervertebral disc prostheses in which the coupling part is mounted on the base part, the one-piece design of the base part together with the coupling part has the advantage that the production method is simplified since operating steps for the separate manufacture of base part and coupling part and the subsequent connection of the two parts can be omitted. The part for the prosthesis can be manufactured as a whole part in one production process This can be carried out using one tool. Moreover, problems concerning the stable and permanent support of the coupling part on the base part, as may arise if the two parts are manufactured separately and then assembled, are avoided as a result of the one-piece design.

As a result of making the base part(s) and the coupling part(s) of one material, it is possible during manufacture to make use only of tools, which can be used to process the material employed. There is no need to use different tools for different materials, and this leads to a cost saving.

One advantage lies in the material properties, that is to say the similar modulus of elasticity to that of cortical bone. In order to further improve the tribological and mechanical properties, it may be provided to use PAEK with a filler material, for example carbon or glass fibres, and/or to modify the polymer matrix, for example by means of crosslinking or iron implantation.

One advantageous embodiment of the invention provides that an anatomically adapted contact surface is formed on a respective outer side of the two base parts. By means of the anatomically adapted contact surface, implantation of the parts assembly as a prosthesis in the skeleton is made possible in such a way that the prosthesis is integrated in the skeleton in as natural a manner as possible. The anatomically adapted contact surface helps the prosthesis to integrate into the skeleton with a precise fit. Slipping of the prosthesis is prevented by means of the anatomically adapted contact surface. Moreover, the arrangement of the bone on the anatomically adapted contact surface when the prosthesis is implanted counteracts any undesirable rotation of the prosthesis relative to the bones, which are adjacent to the prosthesis, so that stable support of the prosthesis is promoted.

It may advantageously be provided that an anti-rotation means is formed on each of the two base parts in order to prevent any rotation of the base parts relative to the bone parts arranged adjacent to the base parts when the prosthesis is implanted. The anti-rotation means preferably comprises a web arranged on the respective outer side of the base parts, wherein openings may be provided in the web. The bone can grow into the openings.

In order to make it possible for the two base parts to move relative to one another, which then makes it possible for the bone parts to move relative to one another when the parts assembly is used in an implanted prosthesis, it may be provided that the two base parts are coupled to one another by means of a sliding connection. The sliding connection is preferably embodied by means of sliding surfaces on the coupling parts. One preferred embodiment of the invention provides that one of the sliding surfaces is formed on a hemispherical protrusion on one of the coupling parts. Once the parts assembly has been assembled, the rounded sliding surface is supported on a countersliding surface on another coupling part, the shape of said countersliding surface being adapted to that of the first sliding surface.

In order to achieve the highest possible abrasion resistance of sliding surface and countersliding surface, these are advantageously coated with a material based on a chromium-nickel alloy.

In one embodiment of the invention, the material used for producing the base parts is preferably a polyetheretherketone. This material has the advantage that a modulus of elasticity is thus provided which is similar to that of cortical bone.

### DESCRIPTION OF PREFERRED EMBODIMENTS OF THE INVENTION

The invention will be explained in more detail below on the basis of preferred embodiments and with reference to a drawing, in which:

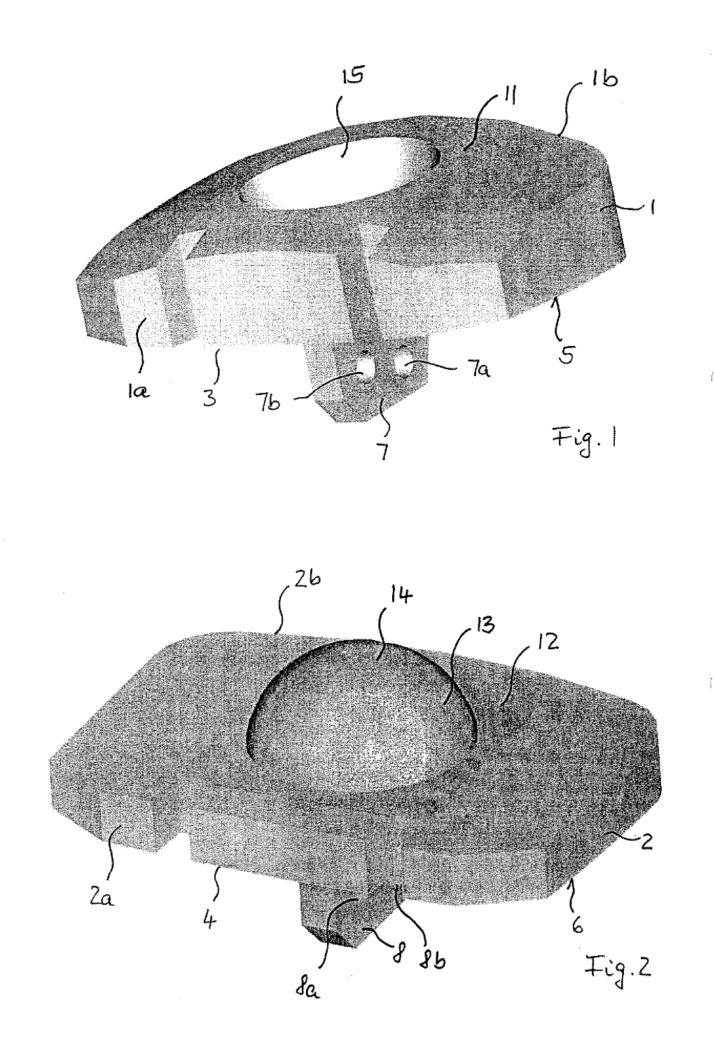

FIG. 1 shows a perspective view of a base part for a parts assembly for use as a prosthesis;

FIG. 2 shows a perspective view of a further base part for a parts assembly, together with the base part of FIG. 1, for use as a prosthesis;

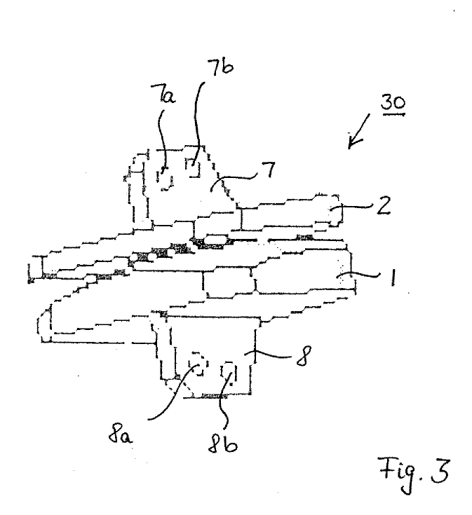

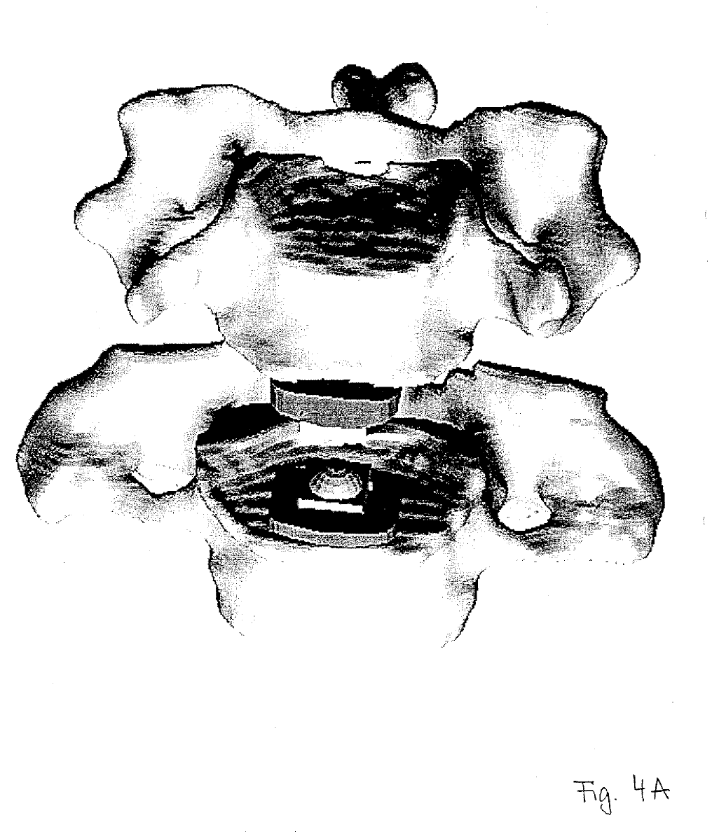

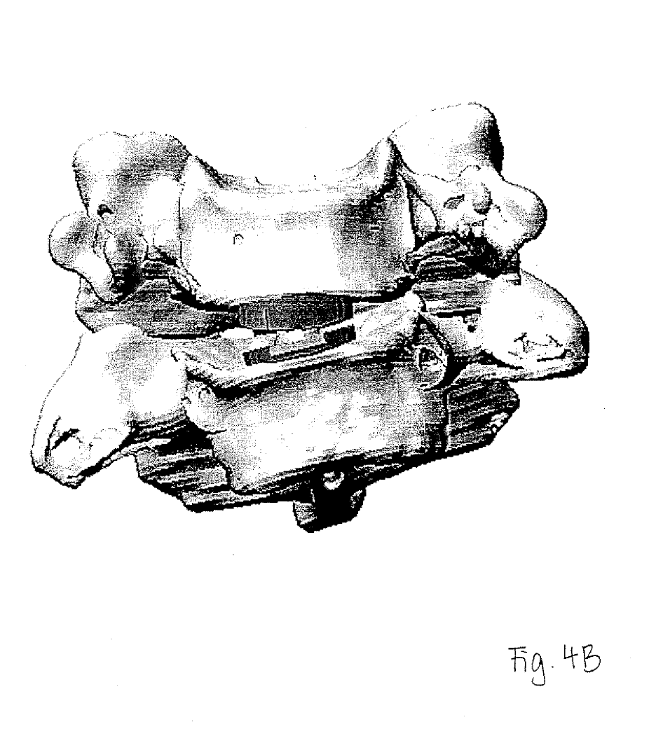

FIG. 3 shows a perspective view of a parts assembly comprising the base part of FIG. 1 and the further base part of FIG. 2 in the coupled state;

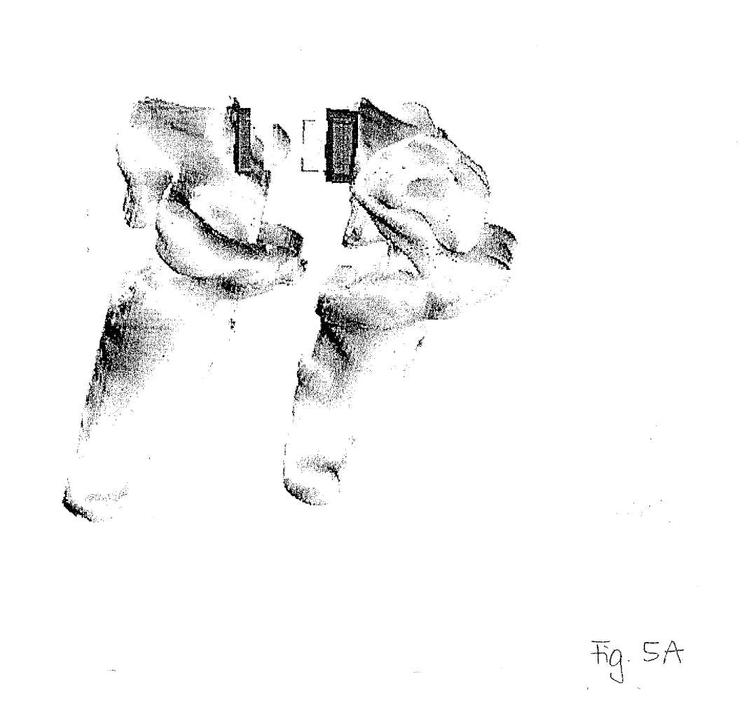

FIGS. 4A and 4B show a view of two bone parts, which are joined by means of a parts assembly according to FIG. 3, in a coupled and decoupled state; and

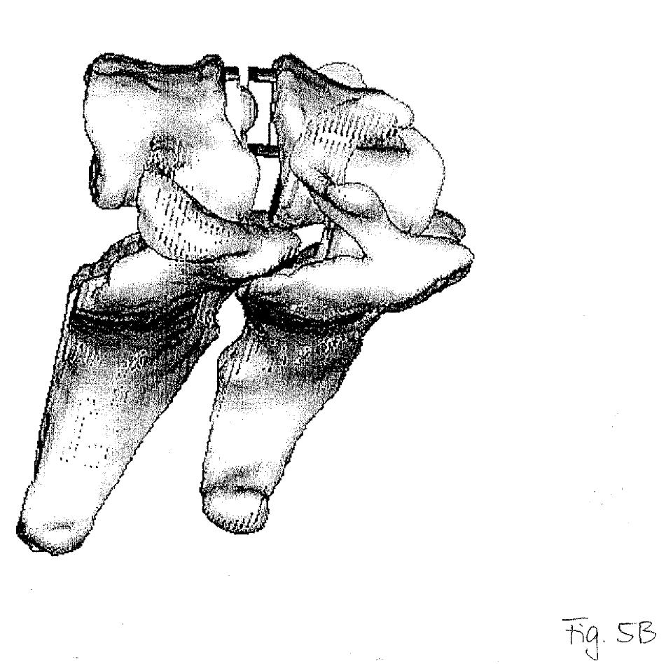

FIGS. 5A and 5B show a view of two further bone parts, which are joined by means of a parts assembly according to FIG. 3, in a coupled and decoupled state.

FIGS. 1 and 2 show a perspective view of a base part 1 and of a further base part 2 for a parts assembly for use as a prosthesis, particularly a cervical spine intervertebral disc prosthesis. FIG. 3 shows a perspective view of a parts assembly 30 in which the base part 1 and the further base part 2 are connected to one another in an articulated manner.

As shown in FIG. 1, the base part 1 has a dorsal section 1a and a ventral section 1b. An anatomically adapted contact surface 5 is formed on an outer side 3. During production of the base part 1, the anatomically adapted contact surface 5 is adapted to the surface contour of the bone in which the prosthesis is to be implanted. When used in connection with an intervertebral disc, the geometry of the contact surface 5 permits as congruent an adaptation as possible to the upper end plates that have been carefully freed from the intervertebral disc cartilage during the operation (intervertebral disc removal).

As shown in FIG. 2, the further base part 2 has a dorsal section 2a and a ventral section 2b. An anatomically adapted contact surface 6 is formed on an outer side 4 of the base part 2. During production of the base part 2, the anatomically adapted contact surface 6 is adapted to the surface contour of the bone in which the prosthesis is to be implanted. When used in connection with an intervertebral disc, the geometry of the contact surface 6 permits as congruent an adaptation as possible to the upper end plates that have been carefully freed from the intervertebral disc cartilage during the operation (intervertebral disc removal).

In order to prevent dislocation of the base parts 1, 2 relative to the bone in an improved manner, a material coating may be provided on the anatomically adapted contact surfaces 5, 6, for example using hydroxylapatite.

As shown in FIGS. 1 and 2, a web 7, 8 is in each case arranged on the outer side 3, 4 of the base parts 1, 2, said web being designed as an anti-rotation means. When the parts assembly is implanted, the respective web 7, 8 engages in a bone depression, so that rotation of the base parts 1, 2 relative to the respectively adjacent bone is not possible. The application of a coating to the webs 7, 8 may be provided in order to prevent dislocation of the prosthesis in situ in an improved manner. The webs 7, 8 have respective openings 7a, 7b and Sa, 8b, through which the bone can grow.

As shown in FIGS. 1 and 2, the base parts 1, 2 have a respective coupling part 11, 12, which is formed in one piece with the associated base part 1, 2. By means of the coupling parts 11, 12, a connection between the two base parts 1, 2 is produced in such a way that the two base parts 1, 2 are connected to one another in an articulated manner. For this purpose, the coupling part 11 shown in FIG. 1 has a protrusion 13, which is shaped in a hemispherical manner. A sliding surface 14 is formed on the protrusion 13, which sliding surface lies on a countersliding surface 15 on the other coupling part 12 once the parts assembly has been assembled (cf. FIG. 3), so that an articulated sliding connection is created between the two base parts 1, 2. By means of this connection, when used as a prosthesis, the parts assembly 30 ensures the mobility and articulation of the skeleton section in which the parts assembly 30 is implanted as a prosthesis.

The base parts 1, 2 and the coupling parts 11, 12 may be varied in terms of their specific design, for example their size, in order to provide different implant sizes and angles, which permit the best possible approximation to the individual anatomy.

The sliding surfaces 14, 15 which provide the articulation are preferably coated with a Co—Cr alloy, which ensures low abrasion under the loads occurring in the cervical spine and thus ensures long-term movement ability.

The two base parts 1, 2, including the coupling parts 11, 12 formed in one piece therewith, are made of polyetherketone (PEK), polyacryletherketone (PAEK), polyetheretherketone (PEEK), polyetherketoneketone (PEKK), polyetherketoneetherketoneketone (PEKEKK) or polyetherketoneetherketone (PEKEK). One advantage of lies in the material properties, that is to say the similar modulus of elasticity to that of cortical bone.

In order to further improve the tribological and mechanical properties, it may be provided to use PAEK with a filler material, for example carbon or glass fibres, and/or to modify the polymer matrix, for example by means of crosslinking or iron implantation.

FIGS. 4A, 4B and 5A, 5B show perspective views of bone parts, which are joined to one another in an articulated manner via a parts assembly 30 as shown in FIG. 3, in a coupled state and a decoupled state, wherein the coupling parts are shown detached from the base parts in FIG. 4A.

The described base parts, in connection with a cervical spine intervertebral disc prosthesis based thereon, lead to the following advantages compared to known prostheses: reconstruction of the segment mobility; reconstruction of the individual cervical spine lordosis by means of possible different angles of the implants; reconstruction of the individual intervertebral disc height by means of possible different implant heights (modular system technology); simplification of the operative procedure for installing the implant; reduction in implant costs due to the use of two materials and modular technology; ability to adapt prosthesis shape to the existing anatomical conditions; no early spontaneous fusion; and implant less susceptible to dislocation.

The instrumentation required for implantation when using a prosthesis based on the described parts can be kept simple and requires for example, in addition to adaptation distractors and an implant holder, only an upper end plate curette and special cervical spine punches, which are able to perform bone decompression on the dorsal side of the vertebral canal even with little distraction of the vertebral motor segment.

The features of the invention, which are disclosed in the above description, the claims and the drawing may be important both individually and in any combination for implementing the invention in its various embodiments.

1. A parts assembly for a prosthesis, particularly a cervical spine intervertebral disc prosthesis, comprising two base parts, which are coupled to one another in an articulated manner by means of coupling parts formed on the base parts, wherein the base parts are in each case formed in one piece with an associated coupling part, wherein the base parts and the coupling parts are made of a material selected from the following group of materials; polyetherketone (PEK), polyetheretherketone (PEEK), polyacryletherketone (PAEK), polyetherketoneketone (PEKK), polyetherketoneetherketoneketone (PEKEKK) and polyetherketoneetherketone (PEKEK).

2. The parts assembly according to claim 1, wherein an anatomically adapted contact surface is formed on a respective outer side of the two base parts.

3. The parts assembly according claim 1, wherein an anti-rotation means is formed on each of the two base parts.

4. The parts assembly according to claim 3, wherein the anti-rotation means comprises a web arranged on the respective outer side.

5. The parts assembly according to claim 1, wherein the two base parts are coupled to one another in an articulated manner by means of a sliding connection.

6. The parts assembly according to claim 5, wherein the sliding connection is embodied by means of a sliding surface formed on one of the coupling parts and a countersliding surface, which is adapted to the sliding surface and is formed on another of the coupling parts, wherein the sliding surface is slidably supported on the countersliding surface in the coupled state of the two base parts.

7. The parts assembly according to claim 6, wherein the sliding surface is formed on a hemispherical protrusion on the coupling part.

8. The parts assembly according to claim 6, wherein the sliding surface and the countersliding surface are coated with a coating material based on a chromium-nickel alloy.

9. The parts assembly according to claim 1, wherein the two base parts and/or the coupling parts are at least partially coated.

10. The parts assembly according to claim 2, wherein the anatomically adapted contact surfaces and/or the webs have a material coating.

11. A part for a prosthesis parts assembly, particularly a cervical spine intervertebral disc prosthesis part, comprising a base part and a coupling part formed on the base part for articulated coupling to another base part, wherein the base part and the coupling part are formed in one piece, and made of a material selected from the following group of materials: polyetherketone (PEK), polyetheretherketone (PEEK), polyacryletherketone (PAEK), polyetherketoneketone (PEKK), polyetherketoneethexketoneketone (PEKKK) and polyetherketoneetherketone (PEKEK).

12. The part according to claim 11, wherein an anatomically adapted contact surface on an outer side of the base part is provided.

13. The part according to claim 11, wherein an anti-rotation means on the outer side of the base part is provided.

14. The part according to claim 13, wherein the anti-rotation means comprises a web arranged on the outer side.

15. The part according to claim 11, wherein a sliding surface is formed on the coupling part.

16. The part according to claim 15, wherein the sliding surface is curved.

17. The part according to claim 15, wherein the sliding surface is coated with a material based on a chromium-nickel alloy.

18. The part according to claim 11, wherein an at least partial material coating of the base part and/or of the coupling part is provided.

19. The part according to claim 12, wherein the anatomically adapted contact surfaces and/or the webs have a material coating.
